(19) United States
(12) Patent Application Publication
(10) Pub. No.: US 2026/0325022 A1
(43) Pub. Date: Oct. 22, 2026

(54) BIOMETRIC MEASUREMENT DEVICES, SYSTEMS, AND METHODS

(71) Applicant: Board of Regents, The University of Texas System, Austin, TX (US)

(72) Inventors: Christopher Klunk, Georgetown, TX (US); Jeffrey Klunk, Warren, MA (US); Iman Salafian, Austin, TX (US); Chris Rylander, Austin, TX (US)

(21) Appl. No.: 19/105,576

(22) PCT Filed: Aug. 25, 2023
(86) PCT No.: PCT/US2023/072895

§ 371 Date: Feb. 21, 2025

Related U.S. Application Data
(60) Provisional application No. 63/373,577, filed on Aug. 26, 2022.

(51) Int. Cl. A61B 5/1455; A61B 5/00

(52) U.S. Cl. CPC A61B 5/14552; A61B 5/6825; A61B 5/6831

(57) ABSTRACT

A biometric measurement device comprises a base having a proximal end, a distal end and a length therebetween, the length having a top surface, a light emitter positioned on the surface of the base, a clip body having a proximal end, a distal end and a length therebetween, the length having a top surface and a bottom surface, a connector connecting the clip body to the base, an band having a first end region and a second end region, wherein the first end region is connected to the base and the second end region is releasably connected to the top surface of the clip body, and a light detecting sensor positioned on the band, such that light detecting sensor is underneath the bottom surface of the clip body and opposing the light emitter when the band is releasably secured to the clip body.

### CROSS-REFERENCE TO RELATED APPLICATIONS

This application claims priority to U.S. provisional application No. 63/373,577 filed on Aug. 26, 2022, incorporated herein by reference in its entirety.

### BACKGROUND OF THE INVENTION

It has been estimated that 10% of newborns require some degree of resuscitation immediately following delivery. One of the earliest and most important steps in neonatal resuscitation is determination of the blood oxygen saturation, allowing use of published minute-by-minute blood oxygen saturation targets to guide therapy. Currently biometric measurement devices used in this setting can been cumbersome to apply, especially for new providers, and frequently requiring troubleshooting before meaningful data can be obtained, wasting valuable time. Problems include issues with adhesive materials on wet neonate skin, time spent peeling adhesive backing, time spent orienting the device correctly and aligning the opposing components of the device on opposite sides of the infant's hand of foot, and an inability to apply sufficient pressure to obtain a reading. Thus there is a need in the art for improved biometric measurement devices, systems, and methods.

### SUMMARY OF THE INVENTION

Some embodiments of the invention disclosed herein are set forth below, and any combination of these embodiments (or portions thereof) may be made to define another embodiment.

In one aspect a biometric measurement device comprises a base having a proximal end, a distal end and a length therebetween, the length having a top surface, a light detecting sensor positioned on the surface of the base, a clip body having a proximal end, a distal end and a length therebetween, the length having a top surface and a bottom surface, a connector connecting the clip body to the base, a band having a first end region and a second end region, wherein the first end region is connected to the base and the second end region is releasably connected to the top surface of the clip body, and a light emitter positioned on the band, such that the light emitter is on a bottom surface of the band and opposing the light detecting sensor when the band is releasably secured to the clip body.

In one embodiment, the base at least partially comprises a smooth rod portion. In one embodiment, the smooth rod portion comprises plastic, Nylon, High-Density Polyethylene (HDPE), Polylactic Acid polymer(PLA), Thermoplastic polyurethane (TPU), Polyvinyl Chloride (PVC), Polyoxymethylene (POM or Delrin), and/or Silicon. In one embodiment, the smooth rod portion has a length of 30 mm to 60 mm. In one embodiment, the smooth rod portion comprises an elliptical cross section with a major axis length of 5 mm to 10 mm, and a minor axis length of 3 mm to 7 mm. In one embodiment, the smooth rod portion comprises a circular cross section with a diameter of 3 mm to 15 mm. In one embodiment, the base has a length of 60 mm to 100 mm. In one embodiment, the clip has a length of 50 mm to 80 mm and a width of 5 mm to 30 mm. In one embodiment, the band has an unstretched length of 50 mm to 250 mm, and a width of 5 mm to 30 mm. In one embodiment, a first operating configuration comprises where the first end region of the band is positioned between the base and the clip, and the second end region of the band wraps around the distal end of the clip and engages a band snap pin on a top surface of the clip. In one embodiment, a second operating configuration comprises where the second end region of the band engages a band locking groove positioned proximate to the distal end of the base, and where the clip is removed from the base. In one embodiment, at least one of the base and the band further includes at least one or more sensors comprising a temperature sensor, an ECG electrode, a gyroscope, an accelerometer, a pressure sensor, a blood pressure sensor, a Ph sensor, analyte sensor, blood sugar sensor, carbon dioxide sensor, moisture sensor, or any other suitable vital sign sensors or biosensors. In one embodiment, the light emitter comprises an LED, a laser, or a fiberoptic. In some embodiments, the light emitter emits light in a wavelength range of 600 nm to 1000 nm. In one embodiment, the clip comprises a wave spring. In one embodiment, the clip is configured to provide a clamping force of 0 grams to 400 grams, or any force at least suitable to keep the device in position, or any force at lease suitable to minimize light leak from the sensors.

In another aspect, a biometric measurement device comprises a top portion, a bottom portion movably, pivotably, or flexibly connected to the top portion, and at least one sensor positioned on or within the top portion or the bottom portion.

In one embodiment, the top portion comprises a first portion of a clip, including a light emitter surrounded by a padded portion positioned on the surface, and the bottom portion comprises a second portion of a clip, including a smooth rod portion and a light detecting sensor positioned on the surface of the smooth rod portion opposite the light emitter. In one embodiment, the top portion comprises an adjustable stretchable strap including a light emitter on the surface of the strap portion, and the bottom portion comprises a smooth sliding rod, including a light detecting sensor positioned on the surface of the smooth rod portion opposite the light emitter. In one embodiment, the top portion comprises a first portion of a parallel clamp, including a light emitter surrounded by a padded portion positioned on the surface of the upper clamp portion, the bottom portion comprises a second portion of a parallel clamp, including a light detecting sensor positioned on the surface of the lower clamp portion opposite the light emitter, and the device further includes a handle comprising first and second portions and a hinge mechanism connecting the upper and lower handle portions to the first and second parallel clamp portions.

In another aspect, a biometric measurement system comprises a biometric measurement device as described above, and a computing system communicatively connected via wired or wireless means to the biometric measurement device, comprising a processor and a non-transitory computer-readable medium with instructions stored thereon, which when executed by a processor, perform steps comprising supplying light at one or more wavelengths to a patient's hand via the light emitter, measuring transmitted and/or reflected light via the light detecting sensor, and calculating a blood oxygen saturation value based on the measured light.

In another aspect, a biometric measurement method comprises providing the biometric measurement system and device as described above, attaching the biometric measurement device to a patient's hand, supplying light at one or more wavelengths to the patient's hand via the light emitter, measuring transmitted and/or reflected light via the light detecting sensor, and calculating a blood oxygen saturation value based on the measured light.

In another aspect, a biometric measurement device comprises an upper handle portion including a light emitter surrounded by a padded portion positioned on the surface of the upper handle portion, and a lower handle portion flexibly connected to the upper handle portion, including a smooth rod portion and a light detecting sensor positioned on the surface of the smooth rod portion opposite the light emitter, wherein the device comprises a clip.

In another aspect, a biometric measurement device comprises an adjustable stretchable strap portion including a light emitter on the surface of the strap portion, and a handle portion flexibly connected to the strap portion, including a smooth rod portion and a light detecting sensor positioned on the surface of the smooth rod portion opposite the light emitter, wherein the device comprises a strap assembly.

In another aspect, a biometric measurement device comprises an upper clamp portion including a light emitter surrounded by a padded portion positioned on the surface of the upper clamp portion, a lower clamp portion including a light detecting sensor positioned on the surface of the lower clamp portion opposite the light emitter, and a handle comprising an upper handle portion and a lower handle portion, further including a hinge mechanism connecting the upper and lower handle portions to the upper and lower clamp portions, wherein the device comprises a parallel hinged clamp.

In another aspect, a biometric measurement system comprises any of the biometric measurement devices as described above, and a computing system communicatively connected to the biometric measurement device, comprising a processor and a non-transitory computer-readable medium with instructions stored thereon, which when executed by a processor, perform steps comprising supplying light at one or more wavelengths to the patients hand via the light emitter, measuring transmitted light via the light detecting sensor, and calculating a blood oxygen saturation value based on the measured transmitted light.

In another aspect, a pulse oximetry method comprises providing the biometric measurement system as described above, attaching the biometric measurement device to a patient's hand via the strap, clamp or clip, supplying light at one or more wavelengths to the patients hand via the light emitter, measuring transmitted light via the light detecting sensor, and calculating a blood oxygen saturation value based on the measured transmitted light.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIGS. 1A-1D depict an exemplary biometric measurement device and system in accordance with some embodiments.

FIG. 2 depicts a first operating configuration (clip configuration) of the exemplary device of FIG. 1 in accordance with some embodiments.

FIG. 3 depicts a second operating configuration (band configuration) of the exemplary device of FIG. 1 in accordance with some embodiments.

FIGS. 4A and 4B depict an exemplary method employing the device and system of FIG. 1 in accordance with some embodiments.

FIGS. 5A-5J depict additional exemplary embodiments of biometric measurement devices in accordance with some embodiments.

FIG. 6 depicts an image of an experimental prototype of the device of FIG. 1 in accordance with some embodiments.

FIGS. 7A-7E depict images of an experimental prototype of the device of FIG. 1 in use in accordance with some embodiments.

FIG. 8 depicts an exemplary experimental result for ECG data acquired via an experimental prototype of the device of FIG. 1 in accordance with some embodiments.

FIG. 9 depicts an exemplary computing environment in which aspects of the invention may be practiced in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of biometric measurement. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are biometric measurement devices, systems and methods.

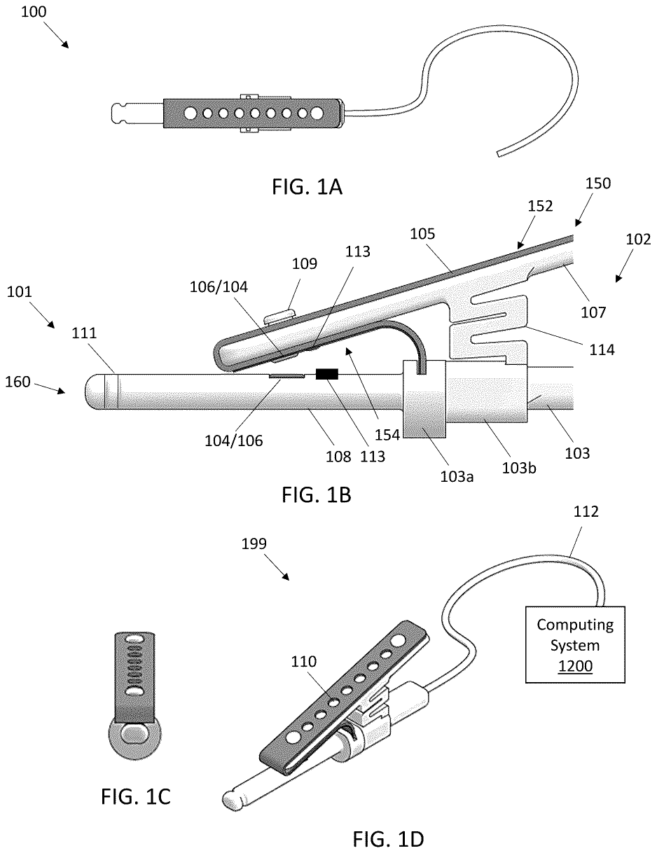

Referring now to FIGS. 1A-1D, 2, and 3, an exemplary biometric measurement device 100 and system 199 is shown. In some embodiments, the device 100 comprises a base 103 having a proximal end 102 and a distal end 101. In some embodiments, the base 103 includes a light emitter 104 and/or light detecting sensor 106 positioned proximate to a top surface of the base 103. In some embodiments, the light emitter 104 and light detecting sensor 106 are a single unit. In some embodiments, the light emitter 104 is adjacent to the light detecting sensor 106 and may be positioned on a top surface of the base 103.

In some embodiments, connection lines, including power and optical lines for example, leading to the sensors (104, 16, 113) may be positioned within base 103 such as through one or more chambers or channel conduits for all necessary wiring. In some embodiments, one or more of a power source, light source, controllers, or similar can be positioned within base 103. In other embodiments one or more of such items can be located outside base 103 via direct wiring to the sensors (104, 16, 113).

In some embodiments, the base 103 has a length of 60 mm to 100 mm. In some embodiments, the base 103 comprises plastic, Nylon, High-Density Polyethylene (HDPE), Polylactic Acid polymer(PLA), Thermoplastic polyurethane (TPU), Polyvinyl Chloride (PVC), Polyoxymethylene (POM or Delrin), and/or Silicon.

In some embodiments, the base 103 may include a smooth rod portion 108 positioned distally on base 103. In some embodiments, the smooth rod portion 108 is sized and shaped to slide easily into a palm side of a semi-clenched or clenched fist of an infant. In some embodiments, the light emitter 104 and/or light detecting sensor 106 are positioned on a top surface of the smooth rod portion 108.

In some embodiments, the smooth rod portion 108 comprises plastic, Nylon, High-Density Polyethylene (HDPE), Polylactic Acid polymer(PLA), Thermoplastic polyurethane (TPU), Polyvinyl Chloride (PVC), Polyoxymethylene (POM or Delrin), and/or Silicon, and may be flexible, semi-flexible or rigid. In some embodiments, the smooth rod portion 108 has a length of 30 mm to 60 mm. In some embodiments, the smooth rod portion 108 comprises an elliptical cross section with a major axis length of 5 mm to 10 mm, and a minor axis length of 3 mm to 7 mm. In one embodiment, the smooth rod portion 108 comprises a circular cross section with a diameter of 3 mm to 15 mm.

In some embodiments, the device 100 further includes a clip having a body 107 having a proximal end 102 and a distal end 101, a top surface including at least one band snap pin 109, and a bottom surface opposing the top surface of the base 103. In some embodiments, the body of the clip 107 is removably connected to the base 103 at a clip connection point 103b near the proximal end 102 of the base 103. In some embodiments, the clip 107 is attached to the base 103 proximate to the smooth rod portion 108. In some embodiments, the clip 107 is hingedly attached to the base 103 via a spring 114 or other mechanism suitable for holding a portion of clip 107 against base 103 under force or tension. In some embodiments, the clip comprises a wave spring 114 providing a wide range of motion for the clip 107 without yielding the clip material, and provides for a low force versus displacement of the clip 107.

In some embodiments, the clip is configured to provide a clamping force of 0 grams to 400 grams, or any force at least suitable to keep the device in position, or any force at lease suitable to minimize light leak from the sensors. In some embodiments, the spring 114 is at a first (lower) tension when the clip 107 is in a closed or more relaxed state, and the spring 114 is at a second (higher) tension when the clip 107 is in an open or strained state, thus biasing the clip 107 towards the close state.

In some embodiments, the clip 107 has a length of 50 mm to 80 mm and a width of 5 mm to 30 mm. In some embodiments, the clip 107 comprises plastic, Nylon, High-Density Polyethylene (HDPE), Polylactic Acid polymer(PLA), Thermoplastic polyurethane (TPU), Polyvinyl Chloride (PVC), Polyoxymethylene (POM or Delrin), and/or Silicon.

In some embodiments, the device 100 further comprises a band 105 having a first end region 152 and second end region 154. In some embodiments, the band 105 is elastic. In some embodiments, the band 105 includes a light detecting sensor 106 and/or light emitter 104 positioned on a bottom surface of the band 105 when band 105 is engaged with clip body 107. In some embodiments, the light emitter 104 and light detecting sensor 106 are a single unit. In some embodiments, the light emitter 104 is adjacent to the light detecting sensor 106 and may be positioned on a bottom surface of the band 105 when engaged with clip body 107. In some embodiments, when band 105 is engaged with clip body 107, the light detecting sensor 106 and/or light emitter 104 are positioned opposite a corresponding light emitter 104 and/or light detecting sensor 106 positioned on the base 103.

In one example embodiment, the light emitter 104 is positioned on the band 105, and the light detecting sensor 106 is positioned on the base 103. In one example embodiment, the light emitter 104 is positioned on the base 103, and the light detecting sensor 106 is positioned on the band 105.

In some embodiments, the first end 152 of the band 105 is connected to the base 103 at a band connection point 103a on the base 103. In some embodiments, the band connection point 103a is between the rod portion 108 and the clip connection point 114. In some embodiments the sensors (104, 106, 113) on the band 105 are electrically connected to the system 199 via the base 103. In some embodiments, the band 105 is positioned between the base 103 and the clip 107.

In some embodiments, the band 105 has an unstretched length of 50 mm to 250 mm, and a width of 5 mm to 30 mm. In some embodiments, the band comprises plastic, Nylon, High-Density Polyethylene (HDPE), Polylactic Acid polymer(PLA), Thermoplastic polyurethane (TPU), Polyvinyl Chloride (PVC), Polyoxymethylene (POM or Delrin), and/or Silicon.

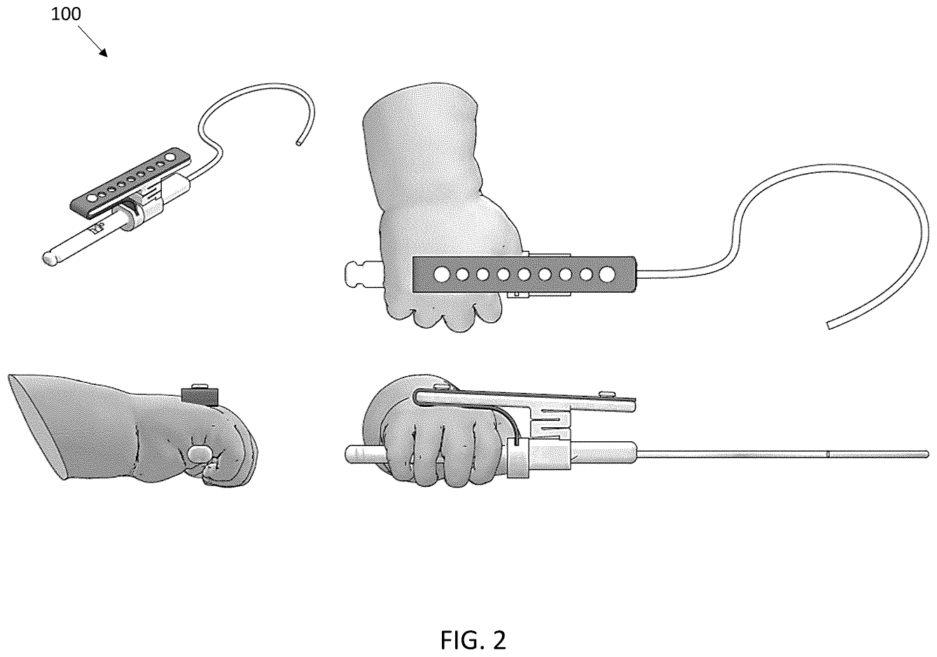

In some embodiments, a first operating configuration (FIG. 2, clip configuration) comprises where a first end region 154 (proximal end) of the band 105 is positioned between the base 103 and the clip 107, and where a second end region 152 (distal portion) of the band 103 wraps around the distal end of the clip 107 and engages via hole 110 a band snap pin 109 on the top surface of the clip 107.

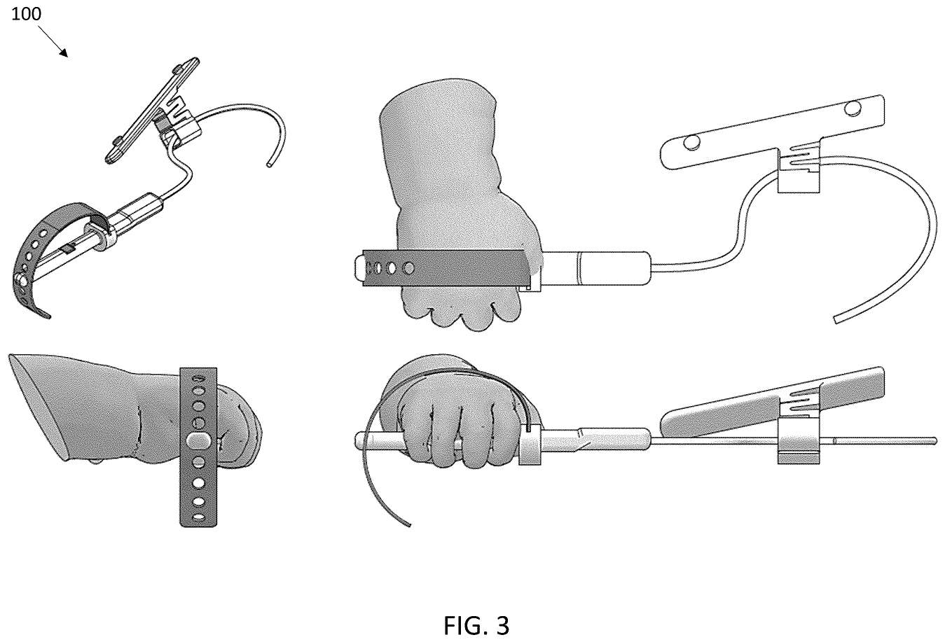

In some embodiments, a second operating configuration (FIG. 3, band configuration) comprises where the second end region 152 (distal portion) of the band 105 engages via one of a plurality of holes 110 a band locking groove 111 positioned proximate to the distal end 101 of the base 103, and where the clip 107 is removed from the base 103.

To switch between configurations, a user may grab the second end region 152 (distal) end of the band 103, stretching the band 103 to remove the band 103 from the band snap pin 109, and selecting a band tension level by selectively engaging one of a plurality of holes 110 of the band 103 with the band locking grove 111 of the base 103.

In some embodiments, the base 103 and/or the band 105 further includes at least one or more additional sensors 113 such a temperature sensor, an ECG electrode, a gyroscope, an accelerometer, a pressure sensor, a blood pressure sensor, a Ph sensor, an analyte sensor, a blood sugar sensor, a carbon dioxide sensor, a moisture sensor, or any other suitable vital sign sensors or biosensors. The additional sensor(s) may be placed anywhere suitable on the device 100.

In some embodiments, light emitter 104 comprises an LED, a laser, a fiberoptic, or similar, can may be configured to output light of one or more wavelengths. In some embodiments, the light emitter 104 emits light in a wavelength range of 600 nm to 1000 nm.

In some embodiments, a biometric measurement system 199 comprises the biometric measurement device 100, and a computing system 1200 communicatively connected via wired or wireless means to the biometric measurement device, comprising a processor and a non-transitory computer-readable medium with instructions stored thereon, which when executed by a processor, perform steps comprising supplying light at one or more wavelengths to a patient's hand via the light emitter 104, measuring transmitted or reflected light via the light detecting sensor 106, and calculating a blood oxygen saturation value based on the measured light.

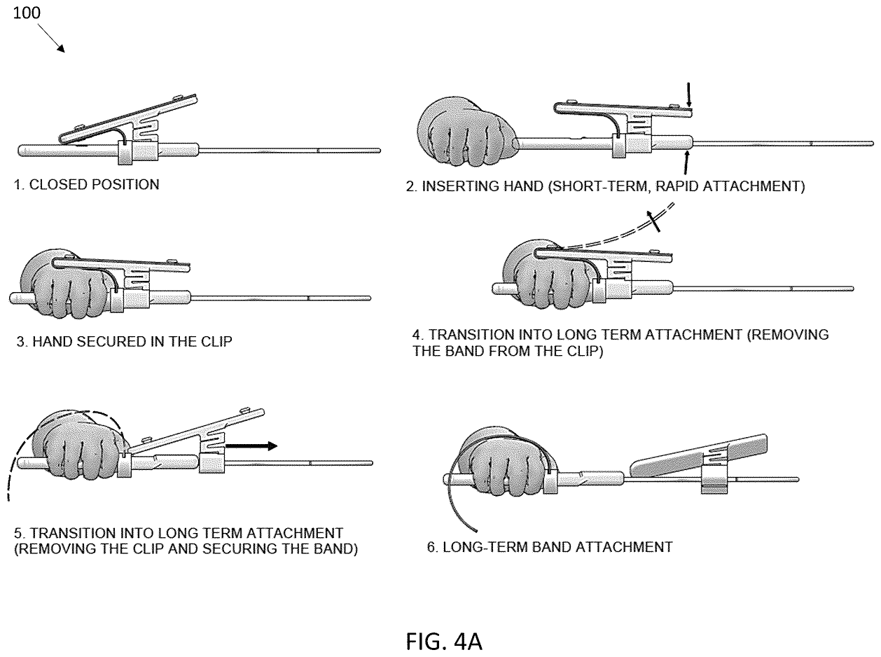

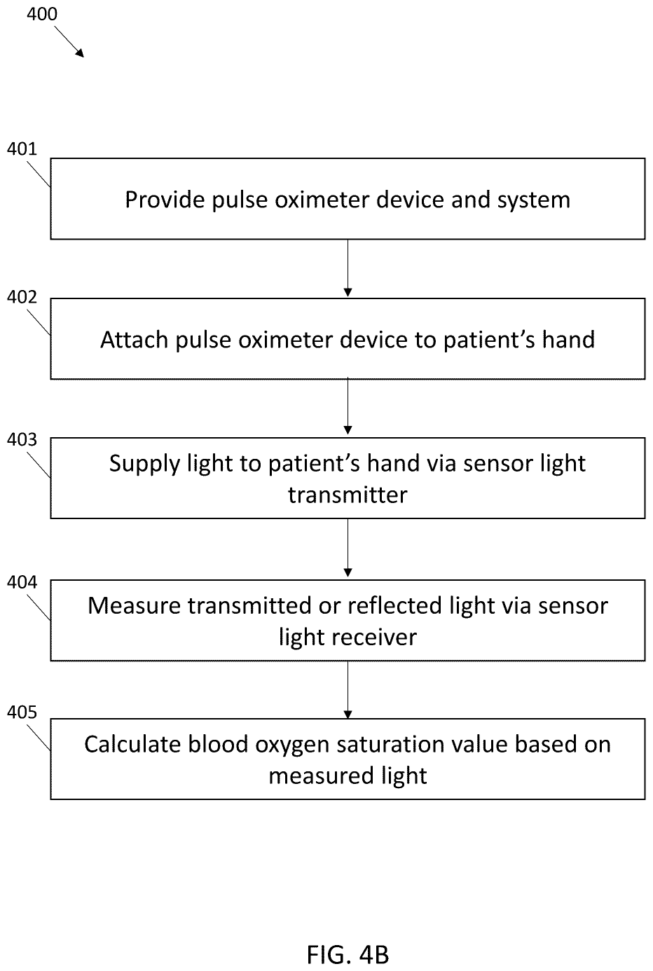

Referring now to FIGS. 4A-4B, a biometric measurement method 400 is shown. At operation 401 the biometric measurement device 100 and system 199 are provided. At operation 402 the device 100 is attached to a patient's hand. The device may be attached via clip, clamp and/or banded means as described herein.

In some embodiments, a rod portion 108 of the base 103 is placed across a palm or inserted into the palm of a semi-clenched fist. In some embodiments, such as for short term and/or rapid monitoring, the clip 107 is used to secure the device 100 to the hand. If longer term monitoring is needed, the device 100 can be transitioned from a clip operating configuration to a band operating configuration by moving the distal end of the band 105 from the top of the clip 107 to the band locking groove 111 on the base 103, and the clip 107 can be removed from the base 103.

At operation 403, light is supplied to the patient's hand via the light emitter 104. The light may be supplied at one or more wavelengths via an LED, bulb, laser, fiberoptic, or similar.

At operation 404 transmitted or reflected light is measured via the light detecting sensor 106. The receiver may comprise a photodiode, photoresistor, or similar.

At operation 405, blood oxygen saturation is calculated based on the measured light.

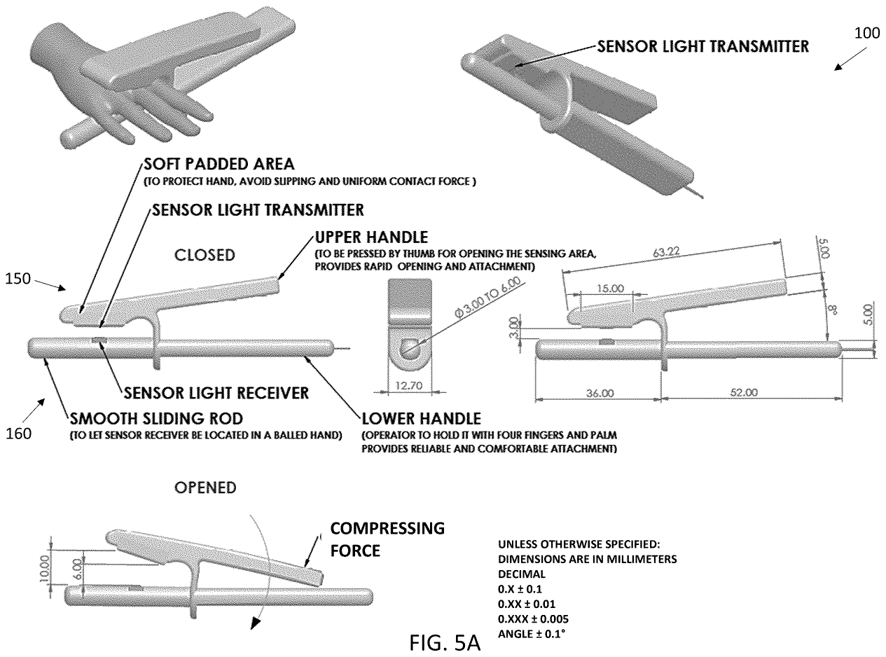

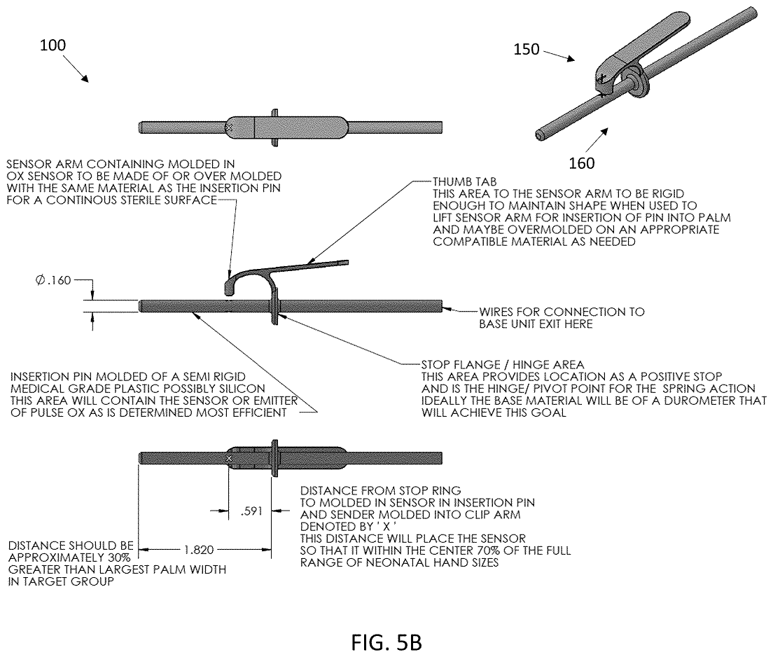

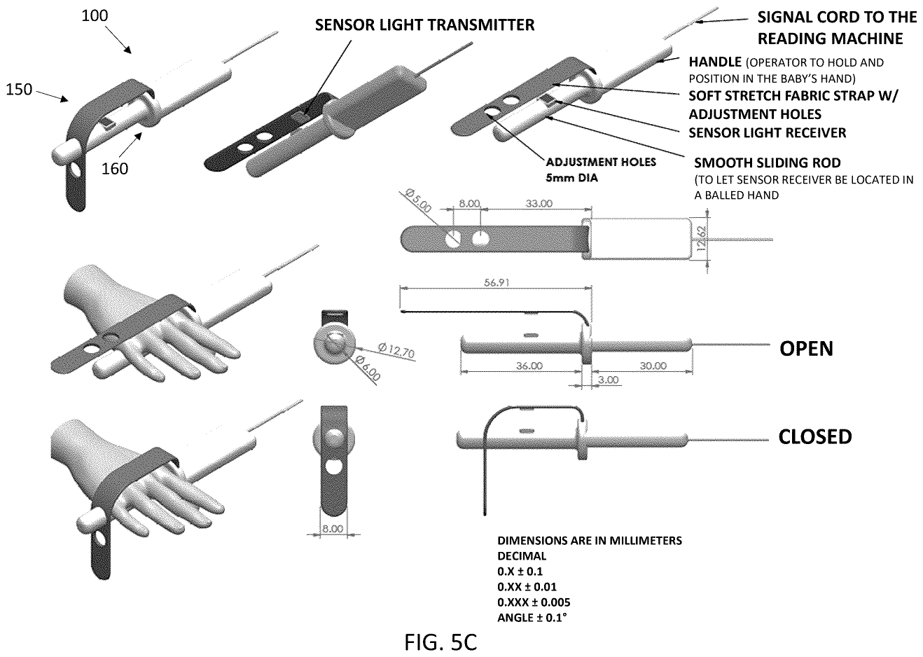

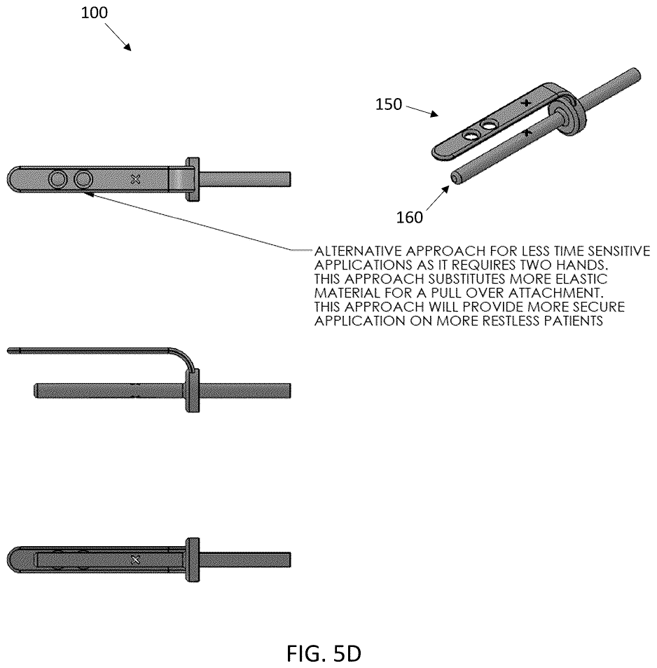

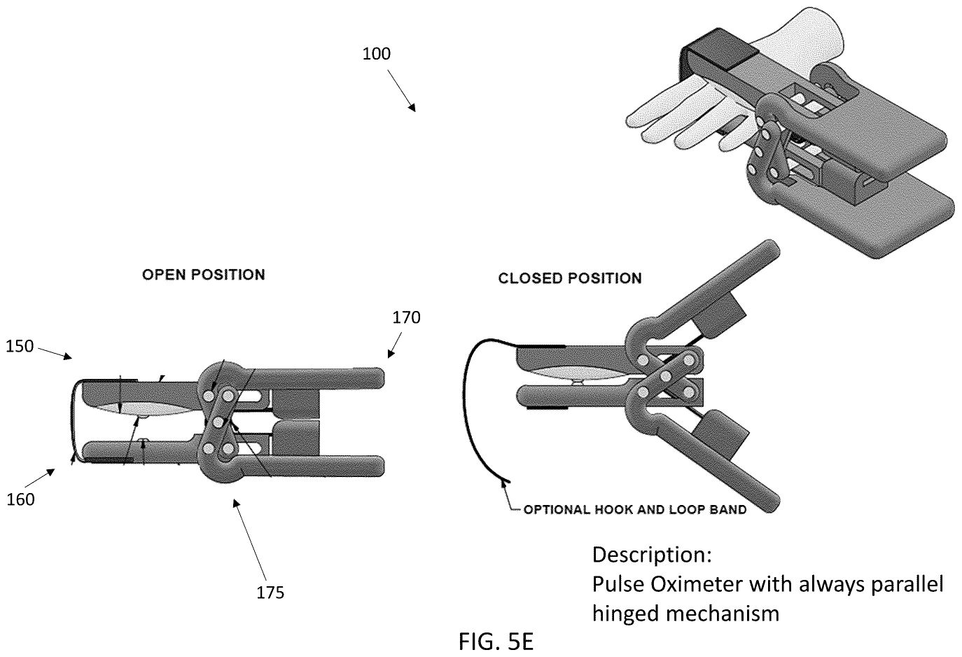

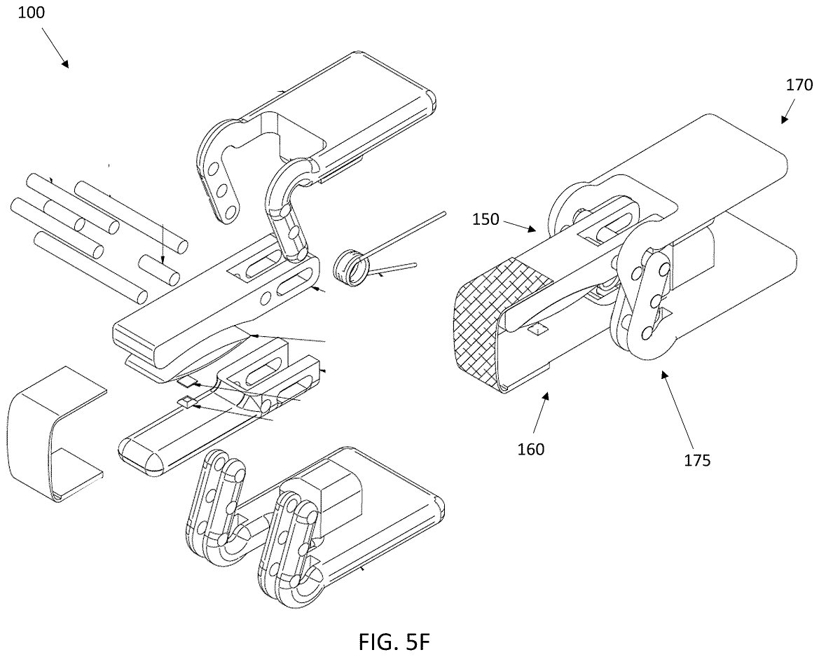

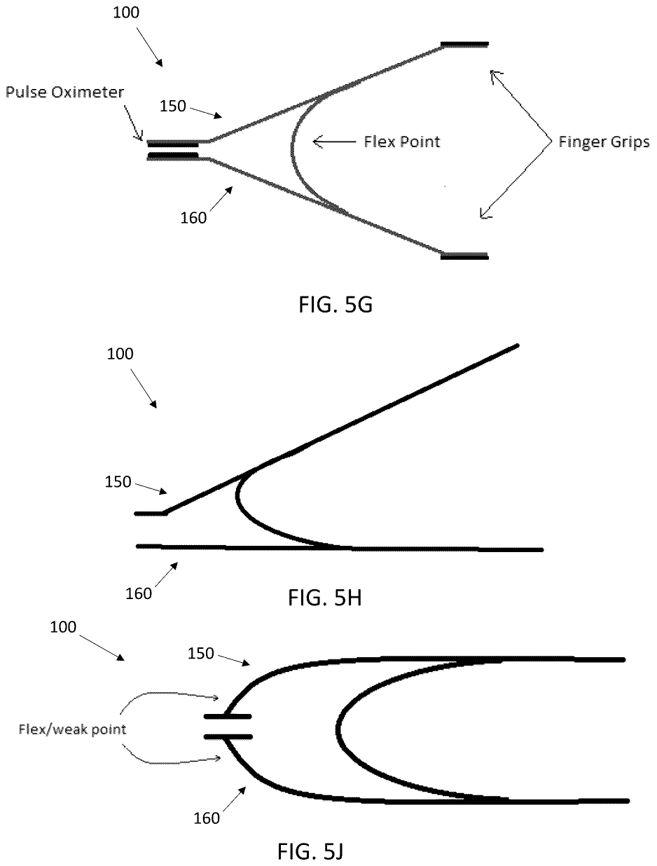

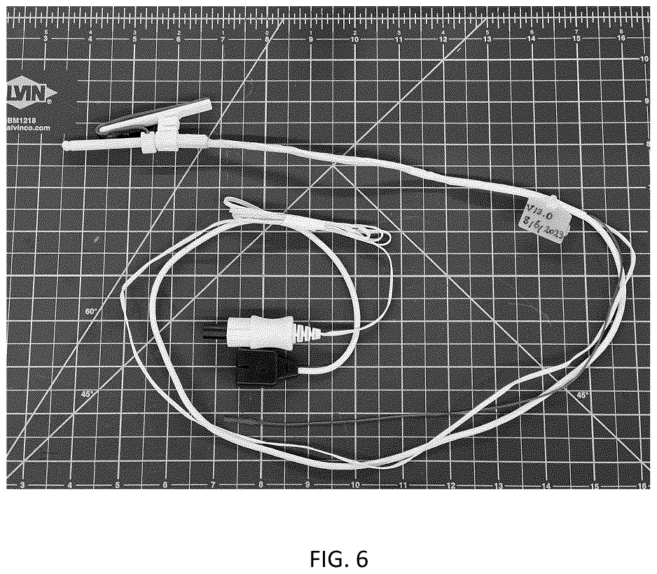

Referring now to FIGS. 5A-5J, additional exemplary embodiments of biometric measurement devices 100 are shown and described. In general, each of the devices comprises a top portion 150, a bottom portion 160 movably, pivotably, and/or flexibly connected to the top portion 150, at least one sensor such as those described above positioned on or within the top portion 150 and/or the bottom portion 160.

In some embodiments, a light emitter 104 is surrounded by a padded portion positioned on the surface of the top portion 150 or bottom portion 160, and a light detecting sensor 106 is positioned on the surface of the portion opposite the light emitter 104.

In some embodiments, the device comprises a clip (FIGS. 5A, 5B, 5G, 5H, 5J). In some embodiments, the device comprises a strap assembly (FIGS. 5C, 5D).

In some embodiments, the device comprises a parallel hinged clamp (FIGS. 5E, 5F) including, in addition to the top and bottom portions (150, 160), a handle 170 comprising an upper handle portion and a lower handle portion, further including a hinge mechanism 175 connecting the upper and lower handle portions to the upper and lower portions (150, 160). In some embodiments, the device optionally includes a hook and loop band configured to secure the clamp closed. In some embodiments, the device further includes a spring for biasing the clamp into an open or closed configuration.

In some embodiments, the device comprises a short-term pulse oximeter. In some embodiments, the device comprises a neonatal pulse oximeter.

In some embodiments, the device is a small clip designed to hold a transmissive pulse oximeter apparatus in one end while allowing a medical/nursing provider to open the device by squeezing the other end, placing it onto the hand of a neonate in need of resuscitation, most frequently immediately after delivery. Accurate blood oxygen saturations are an important piece of data that drives management decisions during resuscitation and the current most commonly used device is cumbersome and inconsistent. The device can be built out of a disposable plastic, though recyclable and reusable materials can be employed.

An easily-applicable clip mechanism can be applied faster than the currently favored pulse-oximeters that involve a device embedded in tape/adhesive material that must have a backing peeled, be oriented with the correct side towards the baby and probes opposing one and other through the baby's extremity, and then often must be secured with an additional strap/device because the adhesive works poorly in the setting of a wet, unclean, newborn. These devices frequently require troubleshooting and often do not provide a reading successfully until several minutes after a decision is made that it is needed. This is due to the time needed for application and issues that arise during the process. The device allows for faster determination of blood oxygen saturations during the critical early moments of a neonatal resuscitation due to faster application because the device can only be applied one way and the two components of the pulse oximeter are always immediately opposing one and other. Furthermore, in some embodiments, there is no adhesive to fail in a wet environment.

The device can be used as a means of obtaining a rapid blood oxygen saturation measurement in any patient small enough for the device to fit. This would allow for use in clinic and emergency department settings. Additionally, a larger device can accommodate patients of any age and size.

In some embodiments, the device comprises a small plastic clip that is closed on one end, at the site of pulse oximeter fixation, and opens when the opposite end is squeezed. This allows for rapid application to a neonate's hand or foot with immediate alignment of the pulse oximeter components. In some embodiments, the small plastic flexible clip is disposable. In some embodiments, the device provides for faster application to the patient with limited potential for user error allowing more rapid determination of a critical vital sign in an unstable patient. The device can be cheap to produce in a disposable form but would allow for later creation of reusable/recyclable versions.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention, and are not to be construed as limiting in any way the remainder of the disclosure.

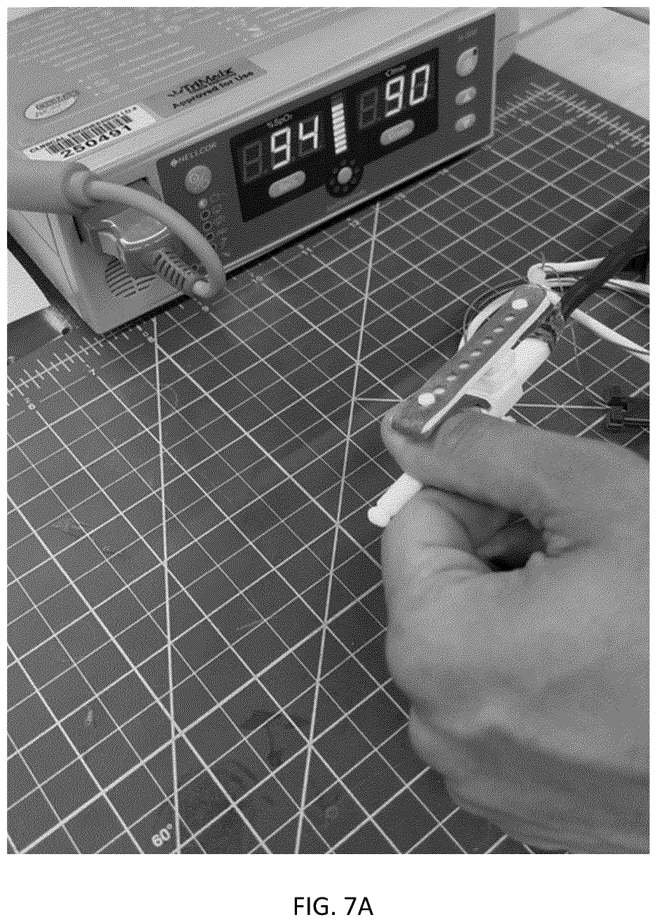

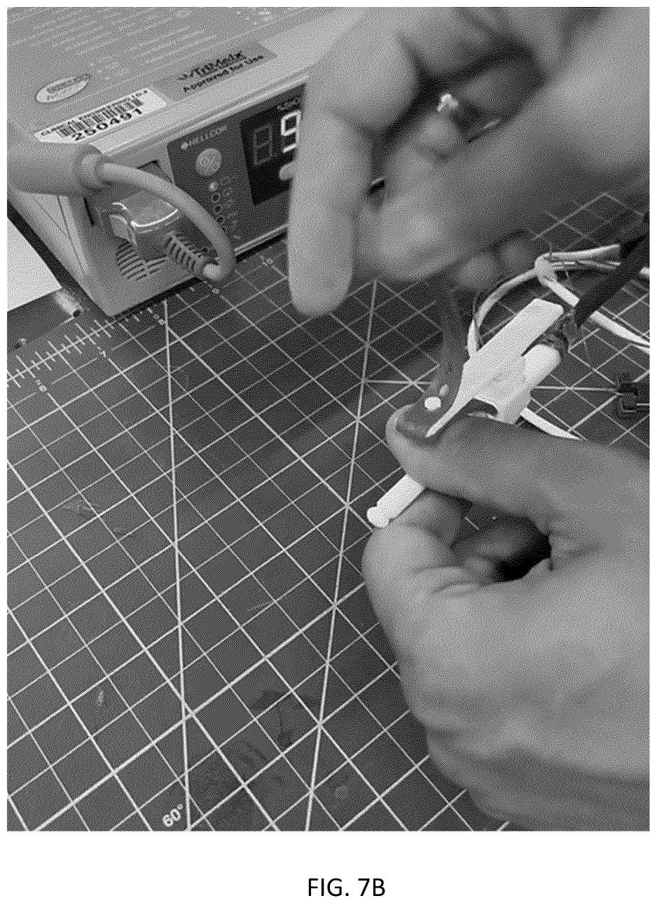

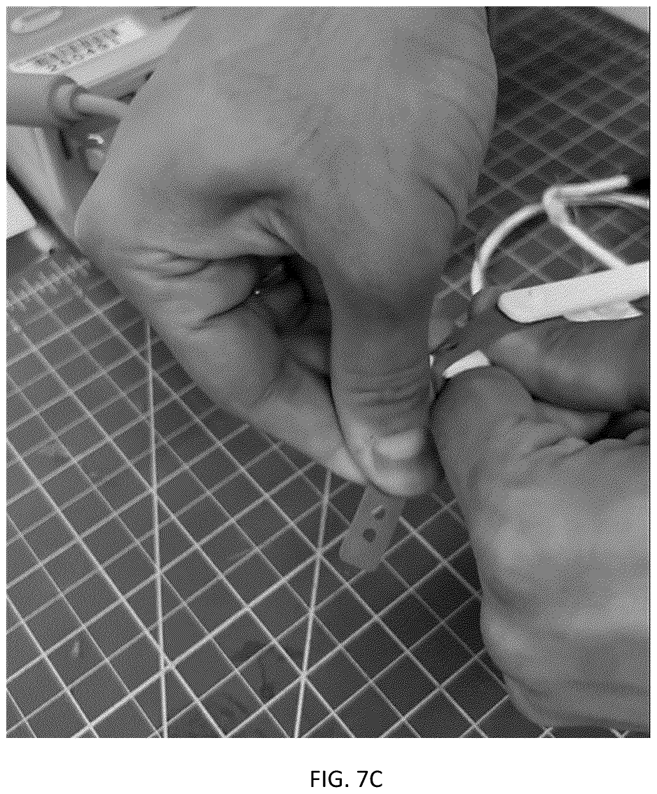

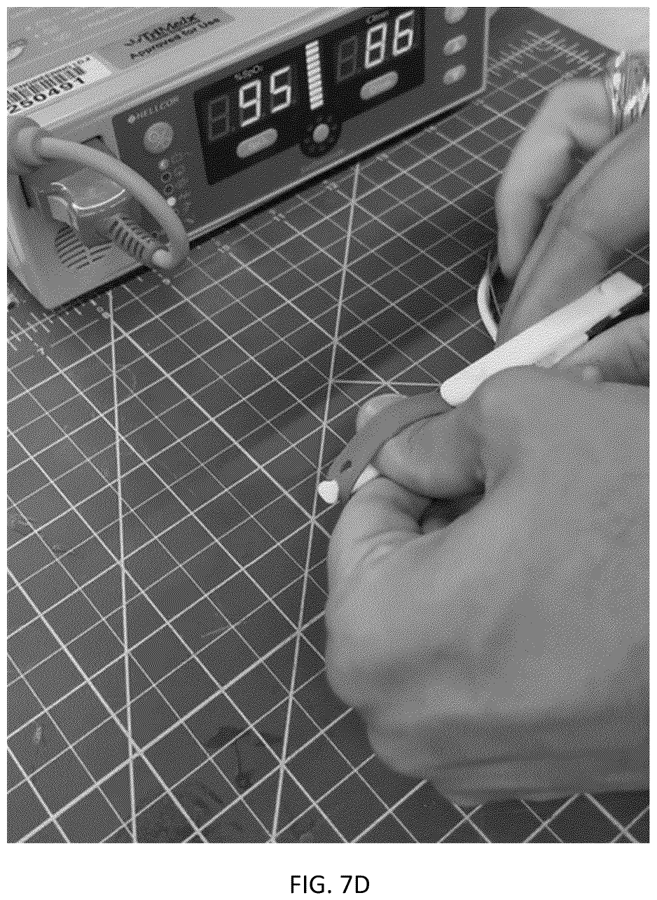

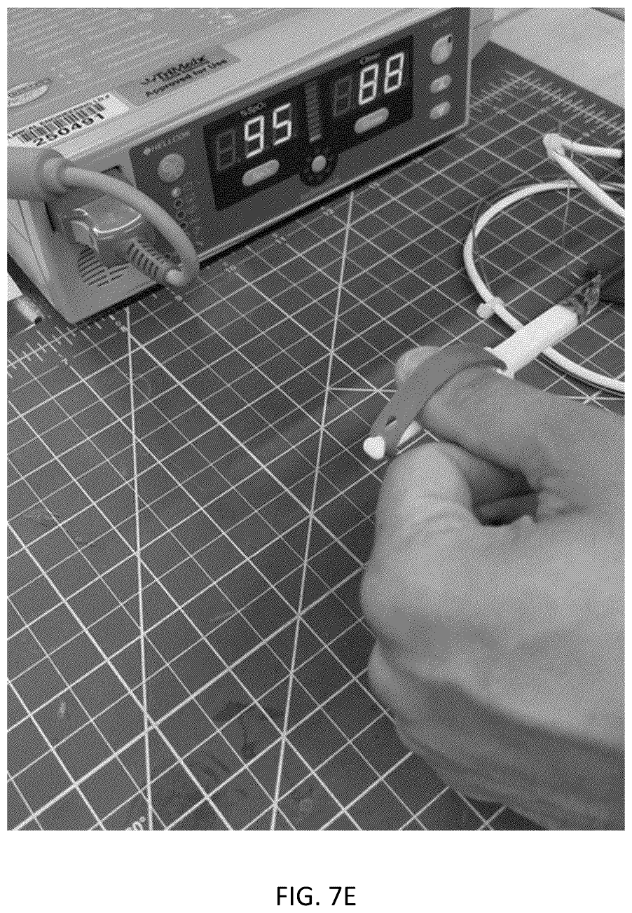

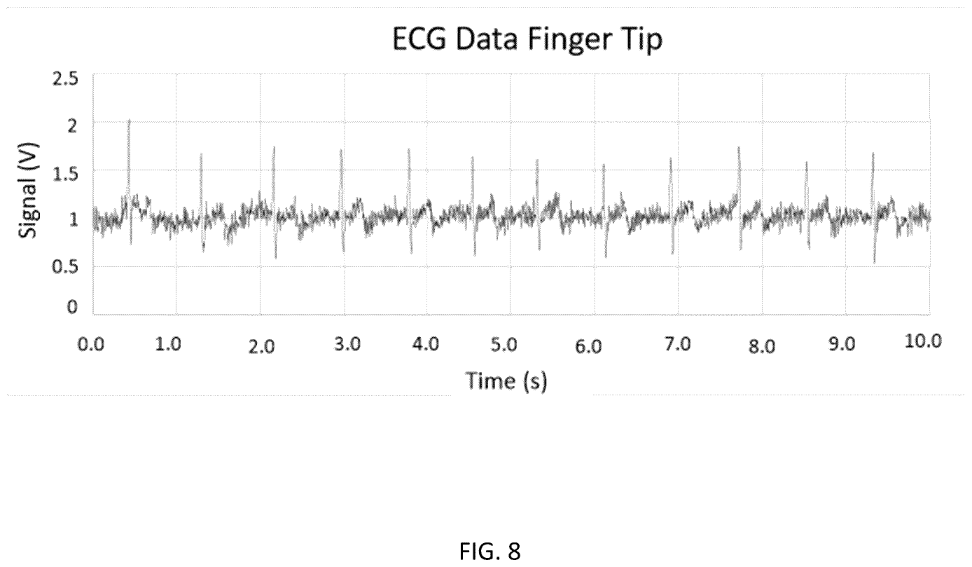

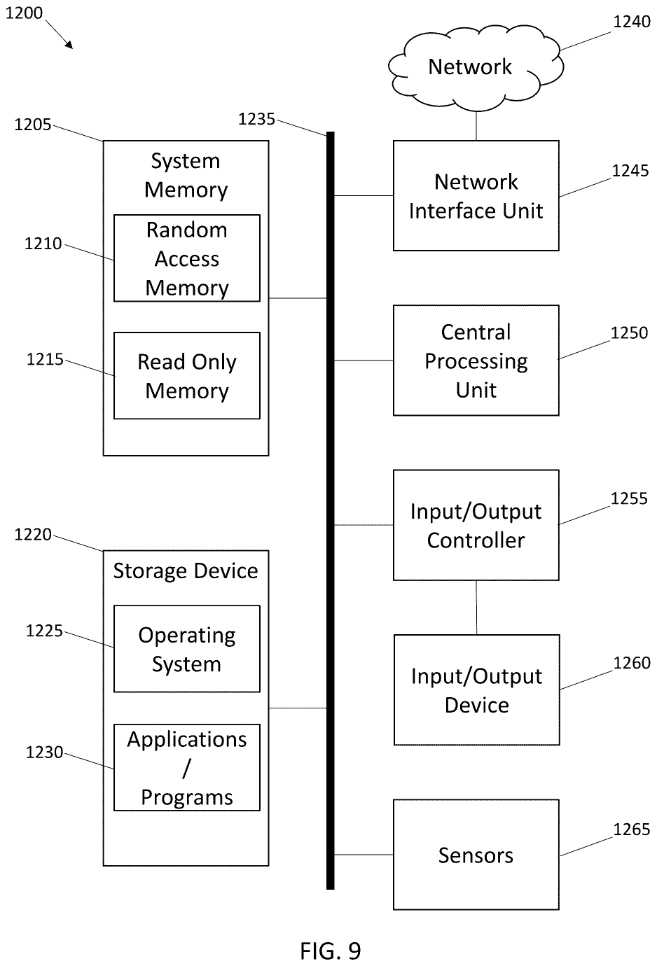

FIG. 6 depicts an image of an experimental prototype of the device of FIG. 1. FIGS. 7A-7E depict images of the experimental prototype in use showing application and reading in a clamp configuration (FIG. 7A), reconfiguration into a band configuration by removing the band from the clip (FIG. 7B) and attaching the band to the locking groove on the base (FIGS. 7C-7D), and removal of the clip (FIGS. 7D-7E). FIG. 8 depicts an exemplary experimental result for ECG data acquired via an experimental prototype of the device.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C #, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

FIG. 12 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 12 depicts an illustrative computer architecture for a computer 1200 for practicing the various embodiments of the invention. The computer architecture shown in FIG. 12 illustrates a conventional personal computer, including a central processing unit 1250 (“CPU”), a system memory 1205, including a random-access memory 1210 (“RAM”) and a read-only memory (“ROM”) 1215, and a system bus 1235 that couples the system memory 1205 to the CPU 1250. A basic input/output system containing the basic routines that help to transfer information between elements within the computer, such as during startup, is stored in the ROM 1215. The computer 1200 further includes a storage device 1220 for storing an operating system 1225, application/program 1230, and data.

The storage device 1220 is connected to the CPU 1250 through a storage controller (not shown) connected to the bus 1235. The storage device 1220 and its associated computer-readable media, provide non-volatile storage for the computer 1200. Although the description of computer-readable media contained herein refers to a storage device, such as a hard disk or CD-ROM drive, it should be appreciated by those skilled in the art that computer-readable media can be any available media that can be accessed by the computer 1200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1200 may operate in a networked environment using logical connections to remote computers through a network 1240, such as TCP/IP network such as the Internet or an intranet. The computer 1200 may connect to the network 1240 through a network interface unit 1245 connected to the bus 1235. It should be appreciated that the network interface unit 1245 may also be utilized to connect to other types of networks and remote computer systems.

The computer 1200 may also include an input/output controller 1255 for receiving and processing input from a number of input/output devices 1260, including a keyboard, a mouse, a touchscreen, a camera, a microphone, a controller, a joystick, or other type of input device. Similarly, the input/output controller 1255 may provide output to a display screen, a printer, a speaker, or other type of output device. The computer 1200 can connect to the input/output device 1260 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1220 and RAM 1210 of the computer 1200, including an operating system 1225 suitable for controlling the operation of a networked computer. The storage device 1220 and RAM 1210 may also store one or more applications/programs 1230. In particular, the storage device 1220 and RAM 1210 may store an application/program 1230 for providing a variety of functionalities to a user. For instance, the application/program 1230 may comprise many types of programs such as a word processing application, a spreadsheet application, a desktop publishing application, a database application, a gaming application, internet browsing application, electronic mail application, messaging application, and the like. According to an embodiment of the present invention, the application/program 1230 comprises a multiple functionality software application for providing word processing functionality, slide presentation functionality, spreadsheet functionality, database functionality and the like.

The computer 1200 in some embodiments can include a variety of sensors 1265 for monitoring the environment surrounding and the environment internal to the computer 1200. These sensors 1265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

1. A biometric measurement device, comprising:
a base having a proximal end, a distal end and a length therebetween, the length having a top surface;
a light detecting sensor positioned on the surface of the base;
a clip body having a proximal end, a distal end and a length therebetween, the length having a top surface and a bottom surface;
a connector connecting the clip body to the base;
a band having a first end region and a second end region, wherein the first end region is connected to the base and the second end region is releasably connected to the top surface of the clip body; and
a light emitter positioned on the band, such that the light emitter is on a bottom surface of the band and opposing the light detecting sensor when the band is releasably secured to the clip body.

2. The device of claim 1, wherein the base at least partially comprises a smooth rod portion.

3. The device of claim 2, wherein the smooth rod portion comprises plastic, Nylon, High-Density Polyethylene (HDPE), Polylactic Acid polymer (PLA), Thermoplastic polyurethane (TPU), Polyvinyl Chloride (PVC), Polyoxymethylene (POM or Delrin), or Silicon.

4. The device of claim 2, wherein the smooth rod portion has a length of 30 mm to 60 mm.

5. The device of claim 2, wherein the smooth rod portion comprises an elliptical cross section with a major axis length of 5 mm to 10 mm, and a minor axis length of 3 mm to 7 mm.

6. The device of claim 1, wherein the base has a length of 60 mm to 100 mm.

7. The device of claim 1, wherein the clip has a length of 50 mm to 80 mm and a width of 5 mm to 30 mm.

8. The device of claim 1, wherein the band has an unstretched length of 50 mm to 250 mm, and a width of 5 mm to 30 mm.

9. The device of claim 1, wherein a first operating configuration comprises where the first end region of the band is positioned between the base and the clip, and the second end region of the band wraps around the distal end of the clip and engages a band snap pin on a top surface of the clip.

10. The device of claim 1, wherein a second operating configuration comprises where the second end region of the band engages a band locking groove positioned proximate to the distal end of the base, and where the clip is removed from the base.

11. The device of claim 1, wherein at least one of the base and the band further includes at least one or more sensors comprising a temperature sensor, an ECG electrode, a gyroscope, an accelerometer, a pressure sensor, a blood pressure sensor, a Ph sensor, an analyte sensor, a blood sugar sensor, a carbon dioxide sensor, a moisture sensor, a vital sign sensor, or a biosensor.

12. The device of claim 1, wherein the light emitter comprises an LED, a laser, or a fiberoptic.

13. The device of claim 1, wherein the clip comprises a wave spring.

14. The device of claim 1, wherein the clip is configured to provide a clamping force of 0 grams to 400 grams.

15. A biometric measurement device, comprising:
a top portion;
a bottom portion movably, pivotably, or flexibly connected to the top portion; and
at least one sensor positioned on or within the top portion or the bottom portion.

16. The device of claim 15, wherein:
the top portion comprises a first portion of a clip, including a light emitter surrounded by a padded portion positioned on the surface; and
the bottom portion comprises a second portion of a clip, including a smooth rod portion and a light detecting sensor positioned on the surface of the smooth rod portion opposite the light emitter.

17. The device of claim 15, wherein:
the top portion comprises an adjustable stretchable strap including a light emitter on the surface of the strap portion; and
the bottom portion comprises a smooth sliding rod, including a light detecting sensor positioned on the surface of the smooth rod portion opposite the light emitter.

18. The device of claim 15:
wherein the top portion comprises a first portion of a parallel clamp, including a light emitter surrounded by a padded portion positioned on the surface of the upper clamp portion;
wherein the bottom portion comprises a second portion of a parallel clamp, including a light detecting sensor positioned on the surface of the lower clamp portion opposite the light emitter; and
further comprising a handle comprising first and second portions and a hinge mechanism connecting the upper and lower handle portions to the first and second parallel clamp portions.

19. A biometric measurement system, comprising:
the biometric measurement device of claim 1; and
a computing system communicatively connected via wired or wireless means to the biometric measurement device, comprising a processor and a non-transitory computer-readable medium with instructions stored thereon, which when executed by a processor, perform steps comprising:
supplying light at one or more wavelengths to a patient's hand via the light emitter;
measuring transmitted or reflected light via the light detecting sensor; and
calculating a blood oxygen saturation value based on the measured light.

20. A biometric measurement method, comprising:
providing the biometric measurement system of claim 19;
attaching the biometric measurement device to a patient's hand;
supplying light at one or more of wavelengths to the patient's hand via the light emitter;
measuring transmitted or reflected light via the light detecting sensor; and
calculating a blood oxygen saturation value based on the measured light.
